(19) United States
(12) Patent Application Publication
(10) Pub. No.: US 2002/0076561 A1
(43) Pub. Date: Jun. 20, 2002

(54) Solid support for immobilizing oligonucleotides

(76) Inventors: Francoise Vinet, Grenoble (FR); Patrick Chaton, Loutre (FR); Frederique Mittler, St. Egieve (FR); Pierre Barritault, Grenoble (FR)

Correspondence Address: PEARNE & GORDON LLP, 526 SUPERIOR AVENUE EAST, SUITE 1200, CLEVELAND, OH, 44114-1484, US

(21) Appl. No.: 09/997,605

(22) Filed: Nov. 29, 2001

(30) Foreign Application Priority Data
Dec. 14, 2000 (FR) 00 16314

(51) Int. Cl. C23C 16/00; A61L 2/00; B32B 18/00

(52) U.S. Cl. 428/446; 427/2.1; 427/248.1; 427/255.28; 428/702

(57) ABSTRACT

The invention concerns a solid support (1) having a surface (4) for immobilizing oligonucleotides, characterized in that said surface (4) is the surface of a material (3) chosen from among HfO₂, TiO₂, Ta₂O₅, ZrO₂ and a mixture comprising at least one of these materials, said surface (4) having undergone a treatment to make it hydrophilic.

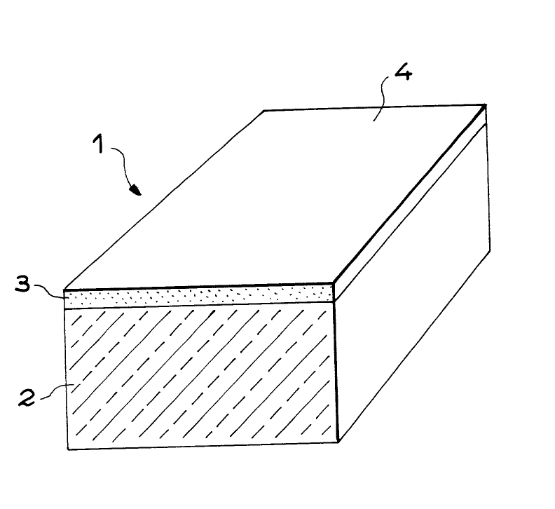

### TECHNICAL FIELD

The invention concerns a solid support for immobilizing oligonucleotides. It also relates to a method for producing said solid support.

The solid support of the invention may be used in particular to produce miniaturized biological assay devices or biochips. These biochips, according to the invention, may be used for example for sequencing, for screening of simple nucleotide polymorphism (SNP), for the study of gene expression, the identification of microorganisms, transcriptome research.

### STATE OF PRIOR ART

Biochips use a technique for grafting oligonucleotides onto a solid support. These oligonucleotides may be obtained by polymerase chain reaction (PCR) which provides DNA fragments of several hundred bases. They may be pre-synthesized, in which case they contain between 6 and 100 parents, or synthesized in situ in which case they contain between 6 and 60 parents.

The fabrication and use of microarrays containing biological probes is part of an area under fast development. Microarrays may be produced by parallel synthesis directly on a solid support. In this case they lead to achieving high density biochips (over 5000 probes). Microarrays may also be produced by immobilizing probes on the surface of the solid support of a biochip. This latter method is more versatile since it allows for the use of both natural and synthetic products which may be purified before immobilization.

The solid supports on which biological molecules are generally deposited are glass, silicon, polyacrylamide gel, polymer supports (see U.S. Pat. No. 5,919,525) or plastic (microwell plates). They may be nylon membranes.

Different techniques may be used for depositing biological compounds on the supports at determined sites. With spotting it is possible to deposit microdroplets on the site: by ink jet, piezoelectric or the so-called “pin and ring” method.

Irrespective of the depositing method used, the solid support must be treated so as to provide attaching or grafting surfaces for the biological molecules. This surface treatment ensures the formation of chemical functions which are generally hydroxyl functions. These functions enable the performance of subsequent chemical grafting steps. In addition, the density of available sites at this step will determine the final density of biological probes on the support.

For substrates in glass or silicon coated with an oxide layer, chemical cleaning of the surface will impart hydroxyl groups to the surface, surface SiO₂ molecules yielding SiOH. Cleaning may be carried out under base conditions (using sodium hydroxide or ammonia) or under acid conditions (using hydrochloric acid, sulfochromic acid or sulfo-oxygenated acid). Measurements made using the droplet angle method are used to characterize the efficiency of conversion of the surface state of the support. The efficiency of the treatment on the immobilization of probes is characterized by hybridisation with complementary probes. Fluorescence observation shows that the more the support surface is hydrophilic, the better the immobilization of the probes, which enables a greater probe density to be obtained on the support.

However, the solid supports of the prior art do not generally have sufficiently satisfactory surface homogeneity for fixing oligonucleotides.

### DISCLOSURE OF THE INVENTION

With the present invention, it is possible to overcome this problem of lack of homogeneity for fixing oligonucleotides.

A first subject of the invention is a solid support having a surface for immobilizing oligonucleotides, characterized in that said surface is the surface of a material chosen from among HfO₂, TiO₂, Ta₂O₅, ZrO₂ and a mixture containing at least one of these materials, said surface having undergone a treatment to make it hydrophilic.

Advantageously, the material is in the form of a layer deposited on a substrate. The thickness of this layer may vary from a few nanometers to one micrometer. The substrate may be a substrate chosen from among substrates in glass, plastic and semiconductor material, silicon for example.

The material having this surface for immobilizing oligonucleotides may be a mixture containing SiO₂.

A second subject of the invention is a biochip containing a solid support for immobilizing oligonucleotides such as defined above.

A third subject of the invention is a method for producing a solid support having a surface for immobilizing oligonucleotides, the support comprising a substrate carrying a layer of material whose free face forms said surface, characterized in that it comprises the following steps:

providing said substrate

depositing on the substrate a layer of a material chosen from among HfO₂, TiO₂, Ta₂O₅, ZrO₂ and a mixture containing at least one of these materials,

treating the free surface of said layer to make it hydrophilic.

The depositing step may consist of depositing a layer of material having a thickness of between a few nanometers and one micrometer.

The substrate-providing step may consist of providing a substrate chosen from among substrates in glass, plastic and semiconductor material.

The depositing step may consist of depositing a material containing SiO₂.

The depositing step may use a deposition method chosen from among vacuum evaporation, ion beam sputtering, radio-frequency sputtering, magnetron sputtering, atom layer vapour phase deposition (ALCVD) and sol-gel deposition.

The treatment step of the free surface of the deposited layer may consist of cleaning the layer with a base solution or an acid solution.

The method may comprise an additional step consisting of structuring the free surface of said layer. This structuring step may use a technique chosen from among dry etching, wet etching and “lift-off”.

### BRIEF DESCRIPTION OF THE DRAWING

The invention will be better understood and other advantages and particular aspects will become apparent on reading the following description given as a non-limitative example, accompanied by the appended drawing which is a perspective view of a solid support having a surface for immobilizing oligonucleotides according to the invention.

### DETAILED DESCRIPTION OF AN EMBODIMENT OF THE INVENTION

The appended figure is a perspective view of a solid support 1 according to the invention. The solid support 1 comprises a substrate 2 in a material enabling the deposition of a layer whose free face is intended to form a surface for immobilizing oligonucleotides. Substrate 2 is for example in silicon.

A layer 3 is deposited on substrate 2. It is used as attaching precursor for the oligonucleotides. It is formed, in whole or in part, of an oxide (or several oxides) of refractory metal, TiO₂, ZrO₂, HfO₂ or Ta₂O₅. The deposited layer is a thin layer having a thickness of between a few nm and 1 μm. This layer may be deposited by vacuum evaporation with electron guns at a temperature of between approximately 50 and 200° C. It may also be deposited by ion beam sputtering (IBS), by radio-frequency or magnetron sputtering. A recent deposition method may also be cited: atom layer chemical vapour deposition (ALCVD). Sol-gel deposition may also be mentioned.

The materials which may form the thin layer of the invention may be deposited by PVD evaporation methods (electron guns, IBS, sputtering), whether on glass, plastic, silicon.

The oxides of these metals are very stable in respect of base solutions, which provides for slow uniform conversion of the surface.

With no surface treatment, these materials are hydrophobic. With hafnium oxide, the water drop angle gives a measurement of 60°. Depending upon the base treatment used, the drop angle varies between 35° to 6°.

Supports treated in this manner were used to grow oligonucleotides by in situ synthesis on a support formed of a layer of HfO₂ on a silicon substrate. On a scarcely hydrophilic sample (35°), the fluorescence signal after hybridisation of probes of 20 parents with complementary targets was weak and non-uniform. A strongly hydrophilic sample (6°) showed a distinct improvement, chiefly in terms of uniformity.

The uniformity obtained was compared with the uniformity of a frequently used solid support, namely a support formed of a silicone substrate coated with a layer of thermal oxide 0.5 μm thick. Compared with this solid support of the prior art, the support of the invention (HfO₂ on Si) provides an improvement in the uniformity of the fluorescence signal.

The same result was obtained with a glass substrate coated with a layer of HfO₂ after immobilization by covalent bonding of probes of 20 pre-synthesized parents.

This type of deposition may also be used for biochips using the probe immobilization method by electrostatic interaction.

The deposition of these refractory metal oxides enables all types of grafting used in biochip technologies, namely all supports (glass, silicon, plastic) and all types of biological grafting techniques (in situ synthesis, immobilization by covalent bonding or electrostatic bonding).

The results of the measurements made show an improvement in the uniformity of the fluorescence signals compared with the prior art.

1. Solid support (1) having a surface (4) for immobilizing oligonucleotides, characterized in that said surface (4) is the surface of a material chosen from among HfO₂, TiO₂, Ta₂O₅, ZrO₂ and a mixture containing at least one of these materials, said surface having undergone a treatment to make it hydrophilic.

2. Solid support according to claim 1, characterized in that the material is in the form of a layer (3) deposited on a substrate (2).

3. Solid support according to claim 2, characterized in that the layer (3) has a thickness of between a few nanometers and one micrometer.

4. Solid support according to either of claims 2 or 3, characterized in that the substrate (2) is a substrate chosen from among substrates in glass, plastic and semiconductor material.

5. Solid support according to claim 4, characterized in that the substrate (2) is in silicon.

6. Solid support according to any of claims 1 to 5, characterized in that said material is a mixture containing SiO₂.

7. Biochip characterized in that it comprises a solid support (1) for immobilizing oligonucleotides according to any of claims 1 to 6.

8. Method for producing a solid support (1) having a surface (4) for immobilizing oligonucleotides, the support (1) comprising a substrate (2) supporting a layer (3) of material whose free face forms said surface, characterized in that it comprises the following steps:
providing said substrate (2),
depositing on the substrate(2) a layer (3) of a material chosen from among HfO₂, TiO₂, Ta₂O₅, ZrO₂ and a mixture containing at least one of these materials,
treating the free surface of said layer (3) to make it hydrophilic.

9. Method according to claim 8, characterized in that the deposition step consists of depositing a layer (3) of material having a thickness of between a few nanometers and one micrometer.

10. Method according to claim 8, characterized in that the substrate (2) providing step consists of providing a substrate chosen from among glass, plastic and semiconductor substrates.

11. Method according to claim 8, characterized in that the deposition step consists of depositing a material containing SiO₂.

12. Method according to claim 8, characterized in that the depositing step uses a deposition method chosen from among vacuum evaporation, ion beam sputtering, radio-frequency sputtering, magnetron sputtering, atom layer chemical vapour deposition (ALCVD) and sol-gel deposition.

13. Method according to claim 8, characterized in that the treatment step of the free surface of the deposited layer (3) consists of cleaning the layer with a base solution or an acid solution.

14. Method according to claim 8, characterized in that it comprises an additional step consisting of structuring the free surface of said layer (3).

15. Method according to claim 14, characterized in that the structuring step uses a technique chosen from among dry etching, wet etching and “lift-off”.
